(19) United States
(12) Patent Application Publication
(10) Pub. No.: US 2024/0123382 A1
(43) Pub. Date: Apr. 18, 2024

(54) FILTER

(71) Applicant: Harada Metal Industry Co., Ltd., Saitama (JP)

(72) Inventors: Mario Harada, Saitama (JP); Moriya Taniguchi, Saitama (JP)

(21) Appl. No.: 17/432,397

(22) PCT Filed: May 21, 2021
(86) PCT No.: PCT/JP2021/019403

§ 371 Date: Aug. 19, 2021

(51) Int. Cl. B01D 37/02; B60H 3/06; C22C 9/02

(52) U.S. Cl. CPC B01D 37/02; B60H 3/0658; C22C 9/02; B01D 2253/1122; B01D 2257/91; B01D 2279/50; B60H 2003/0691

(57) ABSTRACT

A filter loaded with a phosphor bronze alloy powder having antibacterial properties that contains 1.05 wt % tin and 0.09 wt % phosphorus, the remainder being copper and unavoidable impurities.

### FIELD OF INVENTION

The present invention relates to a filter loaded with a phosphor bronze alloy powder having antibacterial properties.

### BACKGROUND ART

It is conventionally known that copper, silver, tin, and the like have antibacterial properties, and they are used in various fields. It is said that the reason why these metals exhibit antibacterial properties is that ions generated by dissolving metals in water destroy the cell walls and cell membranes of microorganisms and bind to enzymes and proteins to reduce their activity and metabolic function. Furthermore, it is said that electrons emitted at the time of ionization generate reactive oxygen species from part of oxygen that is dissolved in air and water, which chemically attack the organic matter in the microorganisms, which is also a factor in antibacterial properties.

Phosphor bronze is an alloy containing tin and recently it is mainly used in electronic parts and various electric products because it has excellent mechanical strength, conductivity, and workability. Phosphor bronze alloy has excellent workability; thus it is compatible with any form and easily processed into forms suiting any purpose, and by successfully utilizing this characteristic and the highly antibacterial properties that this product has, multifaceted expansion of use, which is different from convention, may be expected.

From these viewpoints, if usage examples of copper alloys in a field that require highly antibacterial properties are overviewed, examples include socks to which an athlete's foot prevention effect is imparted by interweaving copper wire therein. Moreover, patent document 2 discloses a filtering device that uses a wire mesh composed of metals such as copper and silver that filters aqueous cleaning solution.

In addition, patent literature 3 discloses an antibacterial deodorant composed of titanium oxide particles loaded with a deodorizing antibacterial component selected from silver, copper, zinc, tin, and the like, and an amine compound. However, none of these are directly touched by human hands and the like; they are spray, mist, or a system placed in a stationary location and operated by pressing only at necessary times and the like, and actually, none can be found that is intended to be touched by hands and requires highly antibacterial properties, such as handrails in a corridor of a medical institution.

### PRIOR ART LITERATURE

### Patent Literature

[Patent Literature 1] JP 2015-214528 A
[Patent Literature 2] JP 2010-137353 A
[Patent Literature 3] JP 2009-268510 A
[Patent Literature 4] JP H5-125591 A

### SUMMARY OF INVENTION

### Problem to be Solved by Invention

Examples of reasons for this include that it had not been clearly shown that phosphor bronze exhibits higher antibacterial properties than pure copper, that the price of copper rose just before around 30 years ago due to being determined by the market price (LME: London Metal Exchange price), not the producer price, that copper alloys readily change color due to contact with the human body, and the like. However, the market price of copper is currently stable.

Therefore, a problem to be solved by the present invention is to exhibit the amplified antibacterial properties of a phosphor bronze alloy powder uniformly applied to a surface of a filter by a spray gun or the like compared to phosphor bronze alloy thin film plate-like products. Furthermore, it is important to verify that the antibacterial properties of the phosphor bronze alloy powder loaded onto the surface are not lost and that the powder is not separated from the surface under any conditions, along with verifying that the antibacterial properties of the phosphor bronze alloy powder are amplified compared to the phosphor bronze alloy thin film plate-like products. Thereafter, a new development in the application of the present product is proposed.

### Means for Solving the Problem

The present invention is conceived to efficiently solve the problem described above. By first clarifying, via a halo test method, the relationship between the antibacterial properties of both a phosphor bronze alloy thin film-applied plate-like product, being the target of comparison, and a phosphor bronze alloy powder of 10 to 20 micrometers, the relationship is also generally tested between the level that antibacterial properties fall when the phosphor bronze alloy powder is added into a mixed solution of an aqueous solution and a binder and uniformly loaded onto a filter by a spray gun, when it is a phosphor bronze alloy thin film-applied plate-like product, and when the phosphor bronze alloy powder is added to a mixed solution of an aqueous solution and a binder and applied by a spray gun or the like.

That is, the present invention is a filter loaded with a phosphor bronze alloy powder having antibacterial properties, containing 1.05 wt % of tin and 0.09 wt % phosphorus, and the remainder being copper and unavoidable impurities.

### Effect of Invention

A filter having antibacterial properties can be provided according to the present invention.

### BRIEF DESCRIPTION OF DRAWINGS

FIG. 1 A photograph illustrating an example of a halo test

FIG. 2 A photograph illustrating an example of a film adhesion test method

FIG. 3A A shape diagram of phosphor bronze alloy powder manufactured by a water atomization method

FIG. 3B A shape diagram of phosphor bronze alloy powder manufactured by a gas atomization method

FIG. 3C A shape diagram of phosphor bronze alloy powder manufactured by a mechanical atomization method

FIG. 4A An outline drawing illustrating a location to place the filter in a wind tunnel test

FIG. 4B An outline drawing representing a wind tunnel used in wind tunnel testing

FIG. 4C An outline drawing illustrating a specific location to place a filter in a wind tunnel test

FIG. 4D A photograph of a filter for 1000 to 1300 cc automobile air conditioning

FIG. 4E A photograph of a filter for 1800 to 2000 cc automobile air conditioning

FIG. 4F A photograph of a filter for 1000 to 1300 cc automobile air conditioning to which phosphor bronze alloy powder is applied

FIG. 4G A photograph of a filter for 1800 to 2000 cc automobile air conditioning to which phosphor bronze alloy powder is applied

FIG. 4H A full-view photograph of the wind tunnel test

FIG. 4I A photograph of a wind tunnel made to accommodate the size of an automobile air conditioning filter

FIG. 5A A diagram of the results of measuring pressure loss in a filter due to applying the phosphor bronze alloy powder

FIG. 5B A diagram of the results of measuring pressure loss in a filter due to applying the phosphor bronze alloy powder

FIG. 6A A photograph for describing a test performed to confirm a level of separation (1)

FIG. 6B A photograph for describing a test performed to confirm a level of separation (2)

FIG. 6C A photograph for describing a test performed to confirm a level of separation (3)

FIG. 6D A photograph for describing a test performed to confirm a level of separation (4)

FIG. 6E A graph showing X-ray fluorescence intensity under five conditions

FIG. 6F A graph of the results of measuring the measuring locations of FIG. 6A (1) to (9) (calculated at n=3, first) under five conditions.

FIG. 6G A graph of the results of measuring the measuring locations of FIG. 6A (1) to (9) (calculated at n=3, second) under five conditions.

FIG. 6H A graph of the results of measuring the measuring locations of FIG. 6A (1) to (9) (calculated at n=3, third) under five conditions.

FIG. 7A A partial view of a nozzle, powder-manufactured using the water atomization method

FIG. 7B A schematic view of a machine performing drying and classifying

FIG. 7C A photograph of a machine that performs drying and classifying

FIG. 7D A size distribution chart (Log diagram) of phosphor bronze alloy powder obtained by classifying water atomized phosphor bronze alloy powder using an air classifier

### EMBODIMENTS OF THE INVENTION

The filter of the present invention is loaded with a phosphor bronze alloy powder having antibacterial properties, containing 1.05 wt % of tin and 0.09 wt % phosphorus, and the remainder being copper and unavoidable impurities.

Here, tin, phosphorus, copper, and unavoidable impurity content in the phosphor bronze alloy powder can be measured by fluorescent X-ray analysis. Energy dispersive X-ray fluorescence spectrometers manufactured by Shimadzu can be given as examples of a measuring device.

Examples of unavoidable impurities include Pb, Be, Co, Si, Ni, S, Zn, Fe, and Al.

X-ray fluorescence analysis is suitable for analysis of components such as powder products and fluid products.

Additionally, scattering of analytical results due to the analytical device and analysis conditions is extremely small in X-ray fluorescence analysis of the phosphor bronze alloy. The present inventors have validated that identical results regarding the composition ratios described in the present specification can be obtained when X-ray fluorescence analysis of two identical test specimens of the phosphor bronze alloy having differing analysis conditions is performed.

In one embodiment of the filter of the present invention, the phosphor bronze alloy powder is added to a mixed solution of an aqueous solution and a binder, stirred thoroughly, then sprayed uniformly onto a surface of the filter, thereby imparting highly antibacterial properties and antiviral properties. The phosphor bronze alloy powder has a size of 10 to 20 micrometers and a convex distribution in grain size.

One embodiment of the filter of the present invention is a filter for household air conditioners or a filter for automobile air conditioners.

One embodiment of the filter of the present invention satisfies the following three elements (1) to (3).

(1) Highly antibacterial properties and antiviral properties are exhibited even at temperatures of 16° C. and 36° C.

(2) The phosphor bronze alloy powder, possessing highly antibacterial properties and antiviral properties even at 30% and 70% humidity, does not separate from the filter, and the metal powder is certainly not sucked inside the vehicle.

(3) The filter is not clogged for at least one year.

In this case, it was able to be confirmed and is shown that the highly antibacterial properties and antiviral properties exhibited or possessed had almost no change when the phosphor bronze alloy powder was uniformly loaded onto a gauze for a mask, instead of a nonwoven fabric, at 266.7 g/m 2 under the following five worse conditions to which the mask is subjected, (i) an initial condition, (ii) conditions after 24 hours, (iii) conditions simulating a fever-like state of someone having a cold or the like, (iv) conditions simulating, in addition to an elevated temperature for the fever-like state, a state of elevated humidity for a runny nose or the like, and (v) conditions of when the mask is removed and places in a bag, suit pocket, or the like.

Here, the phosphor bronze alloy powder not separating from the filter means that the phosphor bronze alloy powder does not separate from the mask gauze under the above conditions (i) to (v) when such is used instead of a filter, and is a result of confirming by X-ray fluorescence intensity that separation does not occur in this softer material. Therefore, the phosphor bronze alloy powder is not orally ingested when someone uses the mask gauze either.

Here, no clogging means that even when the phosphor bronze alloy powder was uniformly loaded onto an upper part of a nonwoven fabric at 224 g/m 2, pressure loss stayed at or below 10% and there was almost no clogging, that is, there was almost no pressure loss.

The size of gauze for a medical mask was determined strategically at either 10×10 cm or 7.5×10 cm, and this was folded eight times or four times.

To prevent the phosphor bronze alloy powder loaded onto the mask from entering the mouth, the 7.5×10 cm type folded eight times was used in the test.

The phosphor bronze alloy powder is not loaded onto an entirety thereof by opening the eight folds, but is loaded onto only one sheet that contacts a main body of the 7.5×10 cm mask.

The medical mask, when folded eight times, is clogged at five micrometers.

Therefore, to keep the phosphor bronze alloy powder loaded onto the medical mask from being orally ingested, the phosphor bronze alloy powder is manufactured by the water atomization method then phosphor bronze alloy powder of five micrometers or less is removed so that by subjecting such to air classification, the phosphor bronze alloy powder classified from the lower limit of 10 to the upper limit of 20 micrometers is used as the phosphor bronze alloy powder for the mask. Thus, the phosphor bronze alloy powder loaded onto the mask is not orally ingested and the highly antibacterial properties thereof are maintained.

Here, the classification is performed based on JIS Z 2510. Specifically, the powder is subjected to the air classifier twice, having the lower limit of 10 micrometers and the upper limit of 20 micrometers, to manufacture the 10 to 20 micrometer product.

The highly antibacterial 10 to 20 micrometer-sized phosphor bronze alloy powder is applied and used in a location generally referred to as a filter, such as a household air conditioning filter and an automobile air conditioning filter, and the intrusion of bacteria and viruses into the household and the automobile can be prevented by the two functions thereof, being the highly antibacterial properties and antiviral properties.

In the process of questioning whether the antibacterial properties are amplified when a phosphor bronze alloy thin film plate product exhibiting highly antibacterial properties is converted into a powdered product by the water atomization method, amplified antibacterial properties were seen compared to the phosphor bronze alloy thin film plate-like product in a test to confirm whether antibacterial effects can be maintained or amplified similarly to the thin film plate-like product when the powder product is applied uniformly to a filter surface of a household air conditioning filter and an automobile air conditioning filter, wherein a phosphor bronze alloy powder was added to a mixed solution of an aqueous solution and a binder, stirred, and uniformly loaded onto a filter surface by a spray gun, and the antibacterial properties of the filter were measured by the halo method.

Meanwhile, pressure loss, which is a necessary function of the filter, raises questions of whether (1) phosphor bronze alloy powder loaded onto a surface brings about a pressure loss great enough to clog venting holes, and whether (2) wind pressure causes separation of the copper powder loaded onto the surface.

The above two questions have been considered and verified for the present product, and it has been demonstrated that such is not the case. A pressure loss test was conducted using an actual filter and a replacement test of harsh surface removal using the mask gauze as an alternative to the filter.

In a comparison of three examples, namely, an example of the antibacterial properties of phosphor bronze alloy powder itself that contains 1.05 wt % of tin and 0.09 wt % of phosphorus, the remainder being copper and unavoidable impurities, an example of the antibacterial properties of a filter wherein the phosphor bronze alloy powder is applied to the filter using a spray gun and the like after combining the phosphor bronze alloy powder, an aqueous solution, and a binder and stirring the mixture solution sufficiently, and finally an example of the antibacterial properties of a thin film plate product which has a different form, an amplification effect emerges for phosphor bronze alloy powder insofar as the specific surface area becomes larger, and antibacterial properties are reduced by 10 to 11% compared to phosphor bronze alloy powder for a filter of which phosphor bronze alloy powder is applied to the surface, but when compared with a thin film product, a roughly 32% increase in the amplification effect is observed. An approximate comparison of these becomes possible using the halo method (however, the number of samples n>3), which is a qualitative analysis method of antibacterial properties. As a remaining problem, there is a need to carefully examine whether phosphor bronze alloy powder that was liquid is peeled during the drying process or starts to elute again when the ambient temperature becomes high, and the like.

Furthermore, one embodiment of the present invention is intended to demonstrate highly antibacterial properties of phosphor bronze alloy powder in two filters, a home air conditioner filter and an automobile air conditioner filter, but by performing experiments to confirm antibacterial properties for the purpose it is important to consider expanding uses in other filters as much as possible in the process.

Moreover, in one embodiment of the present invention, a filter loaded with phosphor bronze alloy powder is obtained by combining a powder product of phosphor bronze alloy powder graded in 10 to 20 micrometers in a mixture of aqueous solution and a binder and spraying on a filter surface (for example, surface of a home air conditioner filter and an automobile air conditioner filter) using a spray gun and the like. viruses and the like are contacted by phosphor bronze alloy powder and killed by antibacterial action while passing through the obtained filter. Killed viruses fall down. The form enables clogging prevention. Furthermore, it has been confirmed that there is no peeling at temperatures about 16° C. to 50° C. and that there is no peeling from the filter even when the humidity becomes 30 to 90% by the power of the binder.

Moreover, one embodiment of the present invention relates to phosphor bronze alloy powder and is a fiber type filter product that is designed to prevent bacteria and viruses from entering or inside home or in an automobile by combining phosphor bronze alloy powder in a mixture of an aqueous solution and a binder and after shaking that sufficiently then uniformly loading the surface of a home air conditioner filter and an automobile air conditioner with such using a spray gun or the like.

The inventors of the present invention repeated tests for antibacterial properties of phosphor bronze alloy powder for antibacterial property relationship with seven different types of filter materials such as acetate fiber, active carbon fiber, and paper filter and the like and additionally examined for conditions; they examined whether each filter could serve its own purpose and role under poor environmental conditions such as the maximum external temperature of 50° C. for automobiles, the minimum internal temperature of 15° C. for automobiles, and the humidity of 30 to 90%. In all cases, nonwoven fabric (paper, felt, and knits) is the main material for home air conditioner filters and automobile air conditioner filters and also it is used under the harshest conditions, so the results of investigation for the nonwoven fabric are mainly shown in the following. Phosphor bronze alloy powder could be uniformly loaded on the surface of the filter and exhibited highly antibacterial properties even on nonwoven fabric. At first, attaching a linear product to a filter in a mesh form was intended, but the test results thereof for antibacterial properties was about 7% (qualitative) of those of a powder product, so the idea was narrowed down to antibacterial properties of phosphor bronze alloy powder and additionally amplifying those.

A factor thought to be one of the factors for the antibacterial properties of copper is that electrons emitted when metal is ionized activate a portion of the oxygen that is dissolved in air and water, as described previously. By differences in ionization potentials and differences in ionization tendencies accompanying such, for phosphor bronze alloy powder, wherein each component contains 1.05 wt % tin and 0.09 wt % phosphorus and the remainder consists of copper and impurities, the reason for antibacterial properties to be higher than phosphor bronze alloy powder of other component compositions from the fact that components constituting the alloy has the ionization potential, which is the maximum and accompanying ionization tendency, which is also the maximum.

The appearance of the phosphor bronze alloy powder is not spherical but in a shape with uneven roughness and is one element for having a maximum specific surface area. That is, as for powders obtained by the water atomization method, the powder product obtained by the machine atomization method has a flat shape, and the powder product obtained by the gas atomization method has a spherical shape; therefore, the specific surface area of powder products obtained by these two methods becomes considerably smaller compared to that of powder products obtained by the water atomization method. Therefore, the inventors selected the water atomization method which maximizes the specific surface area, and found that powdering phosphor bronze alloy by the water atomization method maximizes antibacterial properties of a powder product.

The product of the present invention is an article that is designed to prevent bacteria and viruses from entering and to eliminate fungus odor that cannot be prevented by conventional filters utilizing two functions of phosphor bronze alloy powder, namely, one being highly antibacterial and antiviral properties and the other being deodorizing properties by collecting the phosphor bronze alloy powder in a convex distribution range of a specific size of 10 to 20 micrometers and uniformly loaded with phosphor bronze alloy powder on a filter part such as a home air conditioner filter and an automobile air conditioner filter, that is, an intake and an exhaust part. Also, the characteristics of the present invention enable a use that is even maintenance-free and semi-permanent continuously, while conventional spray type antibacterial, antiviral agents are temporary actions. Further, it is in a superior position in comparison with silver that has the same antibacterial and antiviral properties in terms of cost.

In many of these experiments, it was found that deodorizing properties can be imparted to the home air conditioner filters and automobile filters in addition to highly antibacterial and antiviral properties.

We will examine and review whether this functionality or a similar function can be imparted to other filters (attached to the intake and exhaust locations) or not and we would like to impart these functions to as many articles as possible.

Examples. (1) Vacuum cleaner: there are many vacuum cleaners that state that they are antibacterial and antiviral, but the consumer affairs agency warns that recently there is almost no antibacterial or antiviral vacuum cleaner in circulation, probably since many of those are imported. Moreover, filters for futon dryers are related to filters for vacuum cleaners, and many dryers are not antibacterial or antiviral or capable of eliminating odors from sweat and the like.

(2) Exhaust filter for kitchen range hood: large washable type filters have been common, but recently large range hoods have been installed due to the spread of condominiums and apartments. In this case, there is a need to intake oil, smoke, and odor immediately. There are also many problems such as remaining odor and oil being stuck and impossible to remove. When the odor, oil, and the like remain, harmful insects are likely to enter from the outside.

(3) Also consider commercial and store-type air conditioner filters, air purifier filters, and the like.

In addition to the two filters that led to the success this time, as in the foregoing, filters are almost always used in places that have intakes and exhausts. We would like to develop and propose filters that can be used for these and the like in any way.

Next, phosphor bronze alloy powder was uniformly loaded on a nonwoven fabric such as a home air conditioner filter, an automobile air conditioner filter, and the like, which is a pre-manufacturing stage; then this nonwoven fabric was sheared into sizes according to the application; the original filters attached to the home air conditioner and automobile air conditioner were removed and replaced, and then the experiment was repeated.

There is no home air conditioner filter to which antibacterial material has been applied or which is impregnated with antibacterial materials. However, although there are air conditioner filters for automobiles that contain antibacterial components, the effect thereof almost disappears in about two months. Otherwise, although the description says that there is a deodorizing effect, this effect nearly entirely disappears after about two months.

In contrast to these, for filters uniformly loaded with phosphor bronze alloy powder, the effect thereof is continuous and in addition, a continuous deodorizing effect is also expected in a similar manner.

Firstly, the phosphor bronze alloy powdered product was obtained by redissolving a phosphor bronze alloy thin film plate product, which is a rolled phosphor bronze alloy thin film product that contains 1.05 wt % tin and 0.09 wt % phosphorus and consists of copper and impurities for the remainder having antibacterial properties, and rapidly cooling the dissolved phosphor bronze alloy by the water atomization method. The powdered product was air graded, and only the powder product of convex distribution of 10 to 20 micrometers were used. The reasons for using 10 to 20 micrometers only is because the powder product needs to be prevented from becoming too small and blocking the mesh of the nonwoven fabric or because the shape is uneven, which is a characteristic of powder creation by the water atomization method, and additionally if the particle diameter is uniform, the particle is precisely loaded on the mesh of the nonwoven fabric.

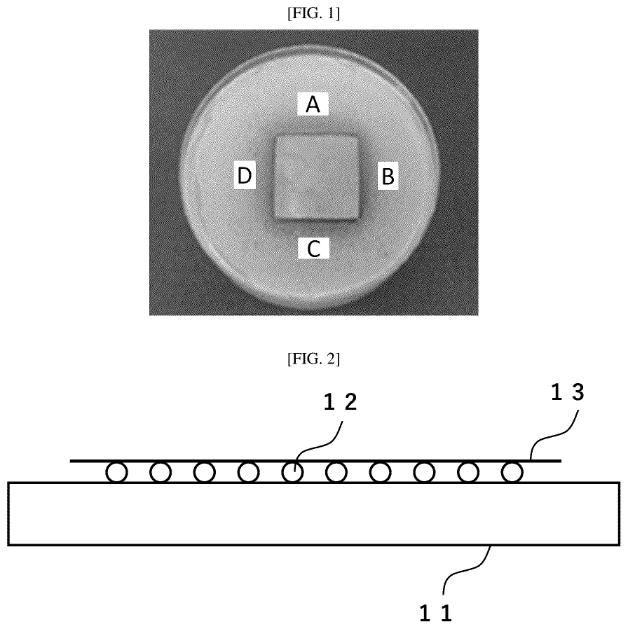

After this highly antibacterial powder product was uniformly loaded on a filter, the filter product was sheared into a length×width of 28×28 mm so that the test sample size thereof was the same as the size of a thin film plate product of a length×width of 28 mm×28 mm, and a halo test in accordance with JIS L 1902 was performed. The bacteria used in the test were one type of Staphylococcus aureus. FIG. 1 is a photograph showing an example of the halo test and an example of Staphylococcus aureus is shown here.

In FIG. 1, the maximum halo widths and averages of the four sides (A, B, C and D) were as follows.
A: 1 mm
B: 1 mm
C: 2 mm
D: 1 mm
Average: 1.25 mm

Phosphor bronze alloy thin film plate product and phosphor bronze alloy powder product exhibit highly antibacterial properties for which a powder product is incomparable.

In the halo test, bacteria were placed into agar (the bait of the bacteria) in a petri dish and cultured; then a specimen of length 28 mm×width 28 mm was placed in the center and maintained for a certain period. Next, the width of the area called the halo where the bacteria around the test piece have disappeared was measured. The test was performed using a different test piece three times per one type of bacteria. As shown by A, B, C, and D in FIG. 1, the halo width was measured on four sides of the test piece, so the n number of test was set to be n=3 this time and the measurement was performed 12 times for the condition.

The film adhesion test method was performed as follows. It should be noted that FIG. 2 is used for the description.

A bacterial solution 12 (0.4 mL) was added dropwise to the surface of a sample 11 (50×50 mm). The test bacteria were Escherichia coli or Staphylococcus aureus.

Next, the viable cell count in the control sample was measured immediately after bacterial solution 12 was added dropwise.

Next, a polyethylene film 13 (40×40 mm) was placed on the bacterial solution 12.

Then, it was stored at 35±1° C., RH 90% or more for 24 hours.

After storage, the viable cell count of the sample 11 was measured.

The increase/decrease value difference was calculated according to the following formula.

Increase/decrease value difference=Log B−Log C

Viable cell count B (antibacterial unprocessed sample)

Viable cell count C (antibacterial processed sample)

The difference in the specific surface area, particle size, and shape of the phosphor bronze alloy powder was confirmed due to the difference in the manufacturing method.

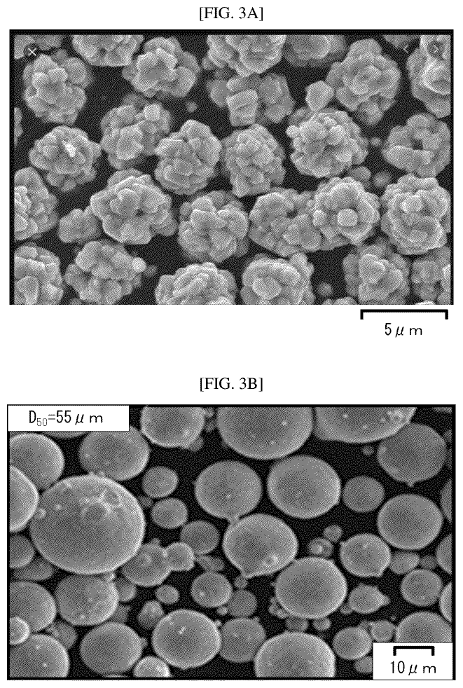

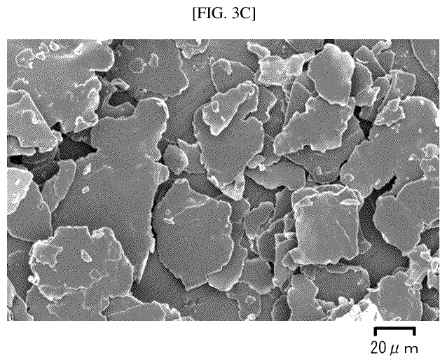

As a conclusion, from FIG. 3A to FIG. 3C, the phosphor bronze alloy powder manufactured by the water atomization method had the largest specific surface area and uniform particle size, as compared with the phosphor bronze alloy powder manufactured by the gas atomization method and the mechanical atomization method.

The shape diagram of phosphor bronze alloy powder manufactured by the water atomization method is shown in FIG. 3A. From FIG. 3A, it is found that there are a large number of protrusions in the phosphor bronze alloy powder manufactured by the water atomization method; that the specific surface area is large; and that it can be well loaded on nonwoven fabric and the like because of the irregularities thereof.

The shape diagram of phosphor bronze alloy powder manufactured by the gas atomization method is shown in FIG. 3B. From FIG. 3B, it is found that the phosphor bronze alloy powder manufactured by the gas atomization method has a spherical shape in addition to different particle sizes, so it is easily understood that the specific surface area is small and also that the particle sizes are different.

The shape diagram of phosphor bronze alloy powder manufactured by the machine atomization method is shown in FIG. 3C. From FIG. 3C, it is found that phosphor bronze alloy powder produced by the machine atomization method has a flat shape, it is difficult to be loaded on the nonwoven fabric, and additionally the particle diameter is large and almost closes the mesh of the nonwoven fabric when it is not loaded. Furthermore, it is found that the pressure loss becomes large.

When the phosphor bronze alloy powder was loaded on an automobile air conditioner filter, the change of the pressure loss of the filter was confirmed. Note that the phosphor bronze alloy powder was loaded on the automobile air conditioner filter by application using a high performance spray gun, and the loaded amount was 127 g/m².

Even if 127 g/m 2 were loaded only on ordinary nonwoven fabric, pressure loss is almost non-existent. This, generally, pressure loss in this degree is within a range which is recognized not to cause any problem. If there is a pressure loss of 20% or more, the filter is just not recognized as an air conditioner filter for automobiles or does not satisfy the use as an air conditioner filter for an automobile.

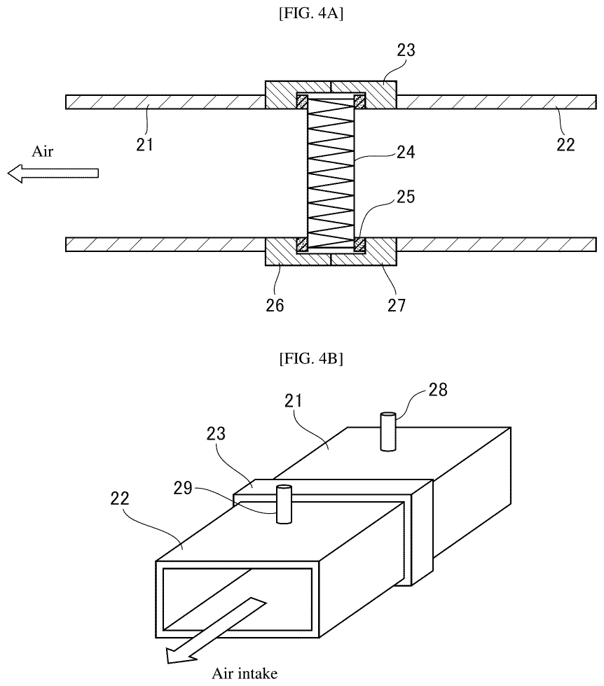

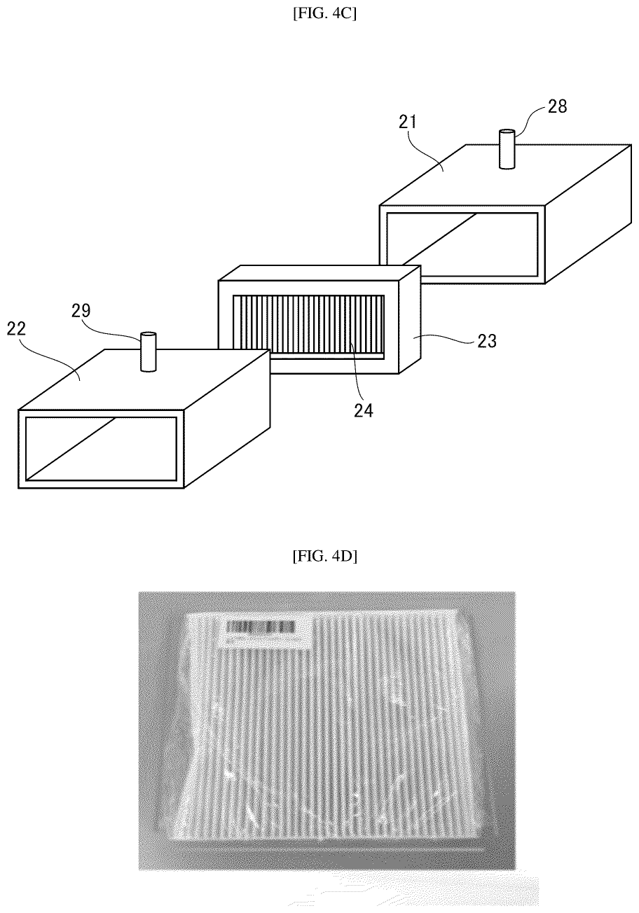

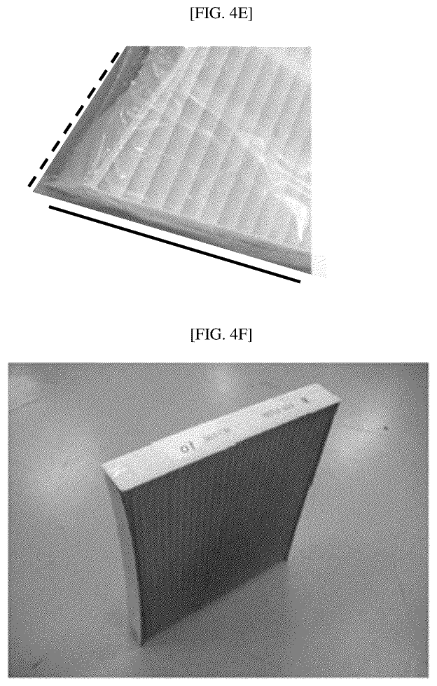

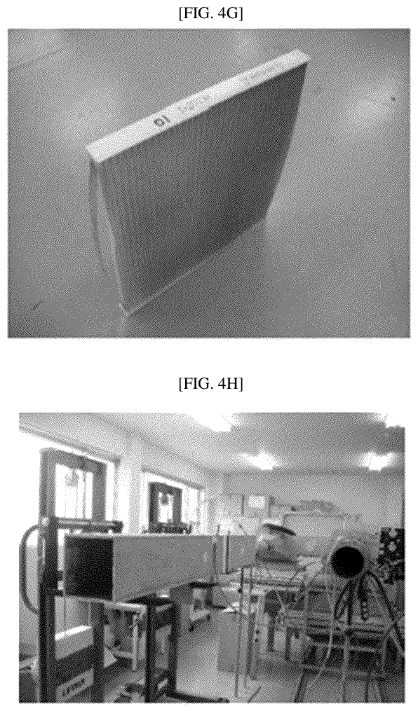

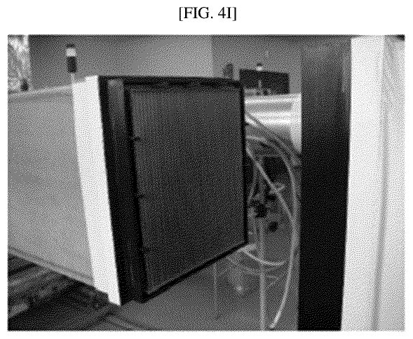

In FIG. 4A to FIG. 4C show the wind tunnel tester used in the wind tunnel test to measure the pressure loss of the filter. Pressurized air was sent into the wind tunnel from the inlet and the pressure was measured at the outlet; then how much the pressure is dropped (pressure loss) is measured.

Here, reference numeral 21 shows an upstream duct. Reference numeral 22 shows a downstream side duct. Reference numeral 23 shows a filter holder. Reference numeral 24 shows a filter. Reference numeral 25 shows a gasket. Reference numeral 26 shows an upstream side holder. Reference numeral 27 shows a downstream side holder. Reference numeral 28 shows a pressure measuring tube.

FIG. 4D and FIG. 4F are air conditioner filters for automobiles of 1000 to 1300 cc. The filter in FIG. 4D is a filter prior to being loaded with phosphor bronze alloy powder, and the filter in FIG. 4F is a filter wherein phosphor bronze alloy powder is uniformly loaded in the filter in FIG. 4D.

FIG. 4E and FIG. 4G are air conditioner filters for automobiles of 1800 to 2000 cc. The filter in FIG. 4E is a filter prior to being loaded phosphor bronze alloy powder, and the filter in FIG. 4G is a filter wherein phosphor bronze alloy powder is uniformly loaded in the filter in FIG. 4E.

FIG. 4H and FIG. 4I are photographs of the overall view of pressure loss of the filters in FIG. 4F and FIG. 4G, on which phosphorus bronze alloy powder is uniformly loaded, using wind tunnels.

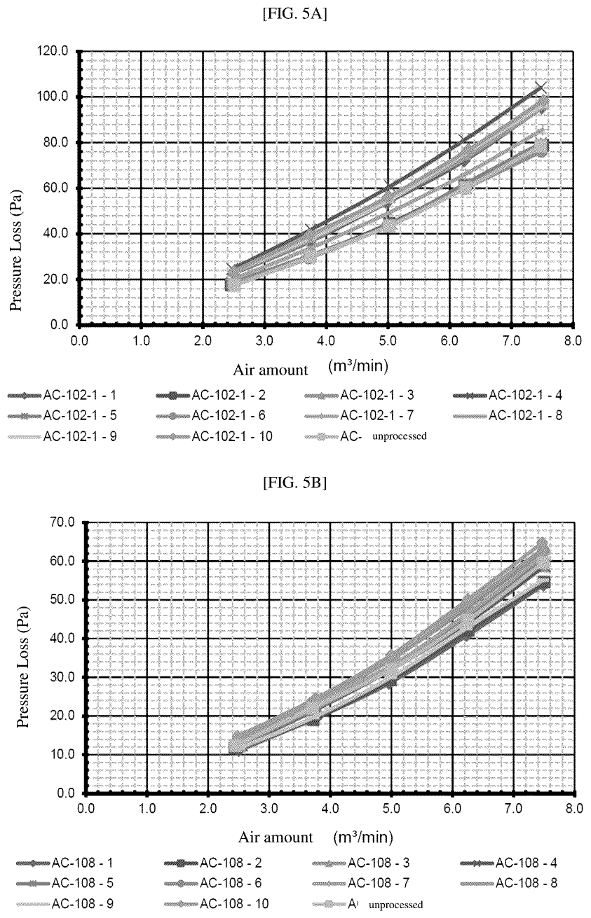

FIG. 5A and FIG. 5B are the results of the actual pressure loss measurements of the filter using the device shown in FIG. 4A to FIG. 4C.

FIG. 5A is the test results of the pressure loss of an air conditioner filter for a 1000 to 1300 cc vehicle. In FIG. 5A, “AC-102-1-1” to “AC-102-1-10” are the measurement results (n=10) of the pressure loss for the filter shown in FIG. 4F, and “AC-102-1-unprocessed” is the measurement result of the pressure loss for the filter shown in FIG. 4D.

FIG. 5B is the test results of the pressure loss of an air conditioner filter for a 1800 to 2000 cc vehicle. In FIG. 5B, “AC-108-1” to “AC-108-10” are the measurement results (n=10) of the pressure loss for the filter shown in FIG. 4G, and “AC-108-unprocessed” is the measurement result of the pressure loss for the filter shown in FIG. 4E.

From FIG. 5A and FIG. 5B, it is found that there is no large change in the pressure loss even when phosphor bronze alloy powder is loaded on the filter.

The size of the air conditioner filter for automobiles of 1000 to 1300 cc is 18 cm in length and 22 cm in width, 1.0 cm in height, and the number of folds is 32.

The size of an air conditioner filter for automobiles of 1800 to 2000 cc is 20 cm in length, 24 cm in width, 1.4 cm in height, and the number of folds is 40.

Phosphor bronze alloy powder was loaded on the gauze for mask, which has a larger degree of freedom than that of an air conditioner filter for automobiles and on which the powder is easily peeled, and the degree of peeling was evaluated. Note that the phosphor bronze alloy powder was loaded on gauze for mask by application using a high performance spray gun and the loaded amount was 266.7 g/m 2.

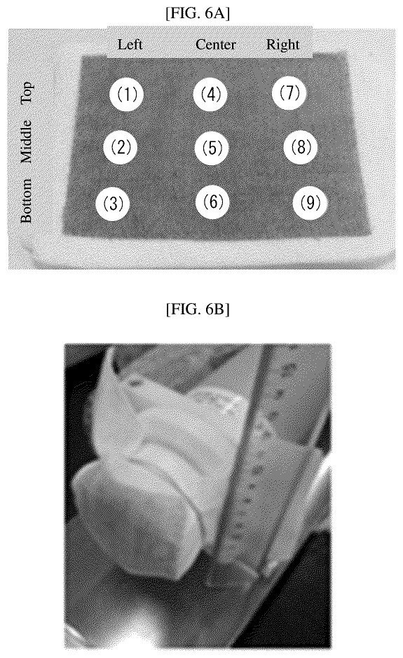

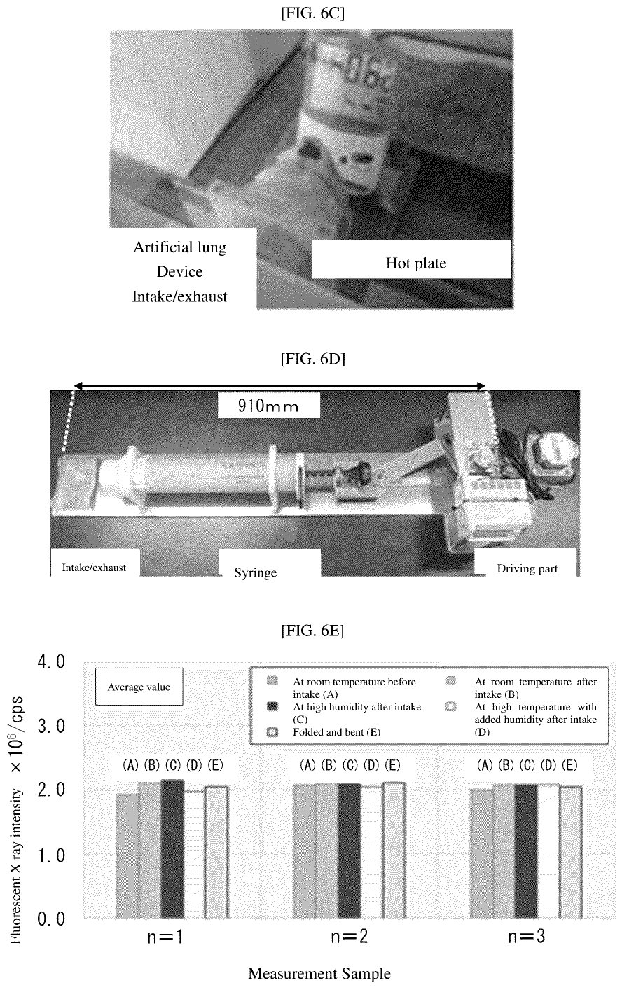

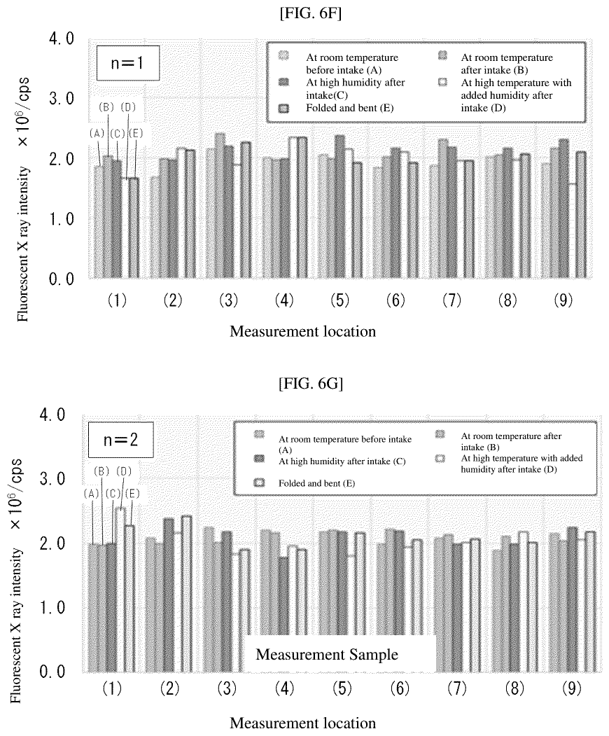

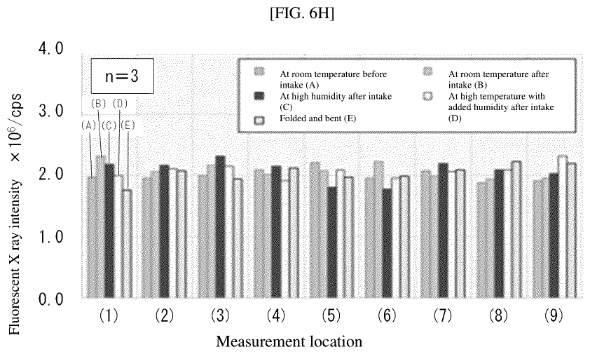

FIG. 6A is a photograph of the gauze for mask, In FIG. 6A, the points indicated by (1) to (9) show the points wherein the degree of peeling was measured. (1) to (9) in FIG. 6A correspond to the measurement points (1) to (9) of the horizontal axis of the graph in FIG. 6F to FIG. 6H.

The instrument shown in FIG. 6B is an instrument similar to an artificial lung. The gauze for mask shown in FIG. 6A is fixed to the opening of a cylindrical plastic having an opening (intake and exhaust opening) of about 12 cm in diameter.

Furthermore, as shown in FIG. 6C, the opening of the instrument shown in FIG. 6B was placed on the top of a hot plate so that the opening was heatable. In addition, by doing so, the intake and exhaust became similar to that of human breathing.

FIG. 6D is a photograph of the overall device. The amount of intake and exhaust passing through the gauze for mask was adjusted so as to be about 500 cc, corresponding to the intake and exhaust air of one human breath. Also the device was designed to change the humidity of the intake and exhaust.

Using the device shown in FIG. 6D, air with adjusted temperature and humidity was passed through the gauze for mask fixed on the opening, and the degree of peeling of the phosphor bronze alloy powder from the gauze for mask was measured. The results are shown in FIG. 6E to FIG. 6H. Note that the degree of peeling was quantified by measuring the intensity of the fluorescent X ray detected by fluorescent X ray analysis. Moreover, the measurement was carried out on three gauzes (measurement samples) for masks loaded with phosphor bronze alloy powder in the same loaded amount.

FIG. 6E is a graph that shows the results of the conditions (A) to (E) on each of three measurement samples (the gauze for mask on which phosphor bronze alloy powder was loaded) Details of conditions (A) to (E) are shown in the following Table 1.

Note that in FIG. 6E, the result of the first measurement sample (n=1) is the average value of measurement locations (1) to (9) in the graph in FIG. 6F.

In FIG. 6E, the result of the second measurement sample (n=2) is the average value of measurement locations (1) to (9) in the graph in FIG. 6G.

In FIG. 6E, the result of the third measurement sample (n=3) is the average value of measurement locations (1) to (9) in the graph in FIG. 6H.

From these results, it can be confirmed that (B) to (E) measured under various conditions compared to the condition (A) before intake and exhaust, have no large change in fluorescent X ray intensity, and that phosphor bronze alloy powder is not peeled from the gauze for mask even after the experiment.

TABLE 1

| Experimental condition | Temperature | Humidity | Remarks |
|---|---|---|---|
| (A) At room temperature | 25° C. | 45% | Before intake and exhaust |
| before intake | | | |
| (B) At room temperature | 25° C. | 45% | Indoor air is taken in and exhausted |
| after intake | | | for 24 hours |
| (C)At high humidity after | 40° C. | 50% | Air simulating a heated state is taken |
| intake | | | in and exhausted for 24 hours |
| (D) At high temperature with | 40° C. | 85% | Air simulating a heated state with |
| added humidity after intake | | | high humidity is taken in and |
| | | | exhausted for 24 hours |
| (E) Folded and bent | 25° C. | 45% | Simulating bending and folding of |
| | | | masks when wearing and taking off |
| | | | and folded/bent 5 times within 8 hours |

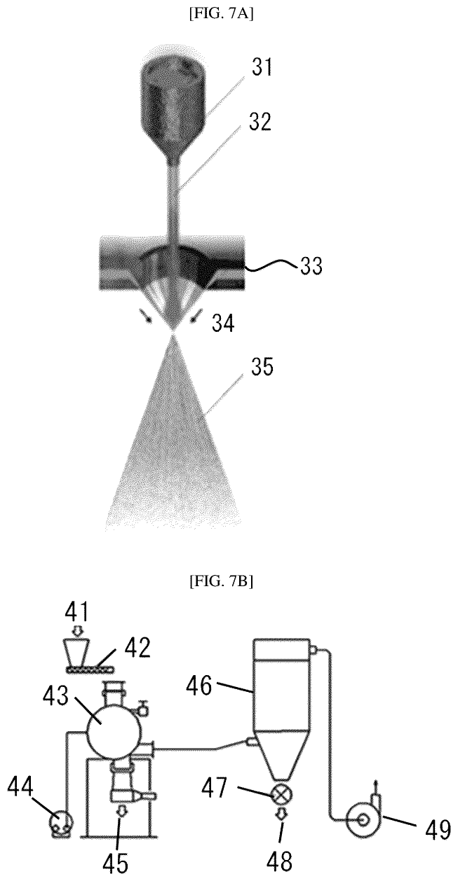

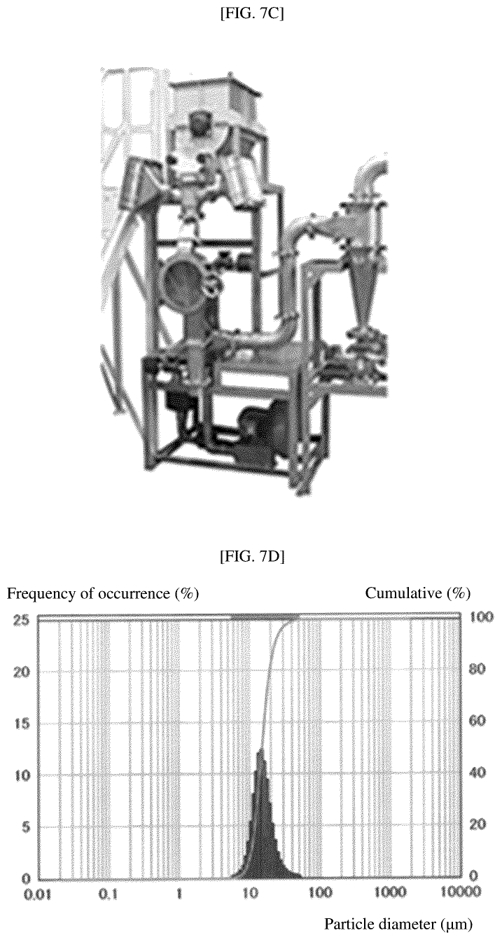

A device shown in FIG. 7A is the core of the water atomization method. The device is one that applies a high pressure cooling water 34, whose pressure is raised to the maximum, from a horizontal hopper water pressure pipe to a vertical lance 33 from around a molten phosphor bronze alloy falling from a lance 32, wherein the molten phosphor bronze alloy flows down from a tundish 31 and passes through the lance 32. A sign 35 shows the form in which finished phosphor bronze alloy powder and water are separated.

Here, in the case of a fine powder, 99.5% or more of the molten phosphor bronze alloy is contained in 0.001 to 140 micrometers.

FIG. 7B is a schematic diagram of a machine that performs drying and grading. A mixture 41 of the phosphor bronze alloy powder obtained by the water atomization method and water enters a cyclone 43 via a belt 42. The cyclone 43 increases speed by a motor 44 and water is discharged from an outlet 45. Then phosphor bronze alloy powder enters a hopper 46. By an air grader 47, heavy objects fall onto an outlet 48 and light objects of the targeted value enter a final grader 49 and then those are regraded and a super fine powder product with the smallest diameter is discharged from an outlet.

FIG. 7C is a photograph of the experiment of a machine that performs drying and grading, which is explained in FIG. 7B,

The table in FIG. 7D is the distribution chart of the phosphor bronze alloy powder obtained by operating the final grader 49 in FIG. 7B compiled with the Log function. It was found that almost all fell into 6 to 50 micrometers. The median value is between 10 micrometers and 20 micrometers, that is, indicating it is around 15 micrometers.

As an example, the table in FIG. 7D is not only an easy-to-understand table of the median value and the like of 10 to 20 micrometers when 10 to 20 micrometers are graded and manufactured by an air grader, but also shows that it is a convex distribution, wherein almost all are in 10 to 20 micrometers, but about 18% (fine powder, coarse powder) are not.

For example, using the device of FIG. 7A, it is investigated at what temperature the phosphor bronze alloy powder has the best yield when it is sprinkled with cooling water.

For example, conditions under which the finished powder product and water can be separated in the water atomization method are determined using the device in FIG. 7B, For example, temperature setting and the like is determined.

According to the present invention, as shown above, the phosphor bronze alloy powder, which has highly antibacterial properties and deodorizing properties, was recognized to have the functions, highly antibacterial properties and deodorizing properties when used in a home air conditioner filter and an automobile air conditioner filter by the above experiments and the antibacterial amplification effect was recognized even when compared with a phosphor bronze alloy thin film plate product and a phosphor bronze alloy linear product and in addition, strong deodorizing properties were recognized in terms of deodorizing properties. With this result it is possible to provide many uses not only for home air conditioner filters and an automobile air conditioner filters but also for other filters, even if filters using nonwoven fabric and paper are used as they are.

It should be noted that the present invention is not limited to the above-described embodiments, and it goes without saying that the present invention includes various variations and modifications that can be conceived by a person having ordinary skill in the art of the present invention, even if there is a design change within a scope that does not deviate from the essential aim of the present invention.

1. A filter loaded with a phosphor bronze alloy powder having antibacterial properties that contains 1.05 wt % tin and 0.09 wt % phosphorus, the remainder being copper and unavoidable impurities.

2. The filter according to claim 1, wherein the phosphor bronze alloy powder has a size of 10 to 20 micrometers, is added to a mixed solution of an aqueous solution and a binder, stirred thoroughly, then sprayed uniformly onto a surface of the filter, thereby imparting highly antibacterial properties and antiviral properties.

3. The filter according to claim 1, the filter being a filter for a household air conditioner or a filter for an automobile air conditioner.

4. The filter according to claim 3, satisfying the following three elements (1) to (3):
(1) highly antibacterial properties and antiviral properties are exhibited even at temperatures of 16° C. and 36° C.;
(2) the phosphor bronze alloy powder, possessing highly antibacterial properties and antiviral properties even at 30% and 70% humidity, does not separate from the filter, and the metal powder is certainly not sucked inside the vehicle; and
(3) the filter is not clogged for at least one year.
